(19) United States
(12) Patent Application Publication
(10) Pub. No.: US 2024/0423944 A1
(43) Pub. Date: Dec. 26, 2024

(54) USE OF LAURYL GALLATE (LG) AS A HEMOSTATIC AGENT

(71) Applicants: CENTRE DE BIOTECHNOLOGIE DE SFAX (CBS), Sfax (TN); INSTITUT NATIONAL DE LA SANTÉ ET DE LA RECHERCHE MÉDICALE, Paris (FR); UNIVERSITÉ TOULOUSE III - PAUL SABATIER, Toulouse (FR)

(72) Inventors: Basma HADJKACEM, Sfax (TN); Ali GARGOURI, Sfax (TN); Marie-Pierre GRATACAP, Toulouse (FR); Jennifer SERIES, Toulouse (FR); Cedric GARCIA, Toulouse (FR); Bernard PAYRASTRE, Toulouse (FR)

(21) Appl. No.: 18/698,509

(22) PCT Filed: Oct. 12, 2022
(86) PCT No.: PCT/EP2022/078424

§ 371 Date: Apr. 4, 2024

(30) Foreign Application Priority Data
Oct. 13, 2021 (TN) 2021/0209

(51) Int. Cl. A61K 31/235; A61K 47/10; A61P 7/04

(52) U.S. Cl. CPC A61K 31/235; A61K 47/10; A61P 7/04

(57) ABSTRACT

The food additive lauryl gallate (LG, E312) is a molecule with antioxidant and hydrophobic properties which has also been shown to exert antibacterial, antiviral and anti-tumoral effects. Here, the inventors show that LG at a low concentration has the ability to spontaneously induce washed human platelet shape change, filopodia emission, granule secretion, phosphatidylserine expression and aggregation. LG was able to activate intracellular signaling pathways including Akt, p38MAP-kinase and calcium response and to trigger activation of the αIIbβ3 integrin. LG also significantly potentiated platelet aggregation induced by low doses of collagen or thrombin receptor agonist peptide. Consistent with this, low doses of LG added to human blood promoted a strong platelet thrombotic response under arterial flow on a collagen matrix. As shown by electron microscopy, at a high concentration, LG induced a dramatic platelet membrane modification associated with calcium influx and a slow platelet aggregation response. Finally, a local flash-application of LG efficiently decreased the tail bleeding in rats suggesting that this compound has the potential to act as a hemostatic. Overall, the results indicate that the food additive LG, possibly through its capacity to modify membrane lateral organization, has pro-aggregant and antihemorrhagic properties.

### FIELD OF THE INVENTION

The present invention is in the field of medicine, in particular haematology.

### BACKGROUND OF THE INVENTION

There is still a need to develop an effective, available and low-cost method to stop bleeding, easy to handle by a non-medical body in an emergency or medical setting.

Lauryl gallate (LG) is the ester of dodecanol and gallic acid (see FIG. 1) and is widely used as an antioxidant food additive under the code E312, and in cosmetics. LG, or dodecyl gallate, is a derivative of gallic acid, an aromatic organic compound that acts as a free-radical scavenger, preventing oxidative rancidity of food, and prevents the generation of superoxide radicals by inhibiting enzymatic peroxidation (Kubo et al., 2002). The low toxicity of this molecule in normal cells justifies its use in the food industry as an antioxidant for more than fifty years (van der Heijden et al., 1986). LG is used as food preservative also because of its antibacterial activity, specifically against Gram-positive bacteria like Bacillus subtilis or Streptococcus mutans, by inhibiting the membrane respiratory chain. LG is also considered as sporicide because it can inactivate spores like those of Bacillus subtilis which are difficult to control due to their intracellular location and resistance to ultraviolet light and heat. Finally, although LG does not have antifungal activity (Kubo et al, 2003), it has a potent antiviral activity (Hurtado et al, 2008). For these different reasons, LG is considered as a helpful molecule in food preservation (Kubo et al, 2003).

In addition, LG has been shown to exhibit anti-proliferative and pro-apoptotic activity in tumoral cell lines with selectivity for rapidly growing cells where it disrupts the mitochondrial membrane potential, activates caspases and induces DNA degradation (Ortega et al, 2003).

However, the effect of LG on hemostasis and thrombosis has not been addressed.

### SUMMARY OF THE INVENTION

The present invention is defined by the claims. In particular, the present invention relates to the use of Lauryl gallate (LG) as a hemostatic agent.

### DETAILED DESCRIPTION OF THE INVENTION

The food additive lauryl gallate (LG, E312) is a molecule with antioxidant and hydrophobic properties which has also been shown to exert antibacterial, antiviral and anti-tumoral effects. The hydrophobic dodecyl tail of LG contributes to its activity by increasing affinity for membrane and acting on the lipid phase transition and likely the lateral membrane organization. Here, the inventors show that LG at a low concentration has the ability to spontaneously induce washed human platelet shape change, filopodia emission, granule secretion, phosphatidylserine expression and aggregation. LG was able to activate intracellular signaling pathways including Akt, p38MAP-kinase and calcium response and to trigger activation of the αIIbβ3 integrin. LG also significantly potentiated platelet aggregation induced by low doses of collagen or thrombin receptor agonist peptide. Consistent with this, low doses of LG added to human blood promoted a strong platelet thrombotic response under arterial flow on a collagen matrix. As shown by electron microscopy, at a high concentration, LG induced a dramatic platelet membrane modification associated with calcium influx and a slow platelet aggregation response. Finally, a local flash-application of LG efficiently decreased the tail bleeding in rats suggesting that this compound has the potential to act as a hemostatic. Overall, the results indicate that the food additive LG, possibly through its capacity to modify membrane lateral organization, has pro-aggregant and antihemorrhagic properties.

The present invention relates to a method of arresting the flow of blood from a bleeding wound in a patient in need thereof comprising the steps of applying a therapeutically effective amount of lauryl gallate (LG).

It is therefore an object of this invention to provide a method of utilizing LG as a hemostatic agent for arresting blood flow from a wound.

As used herein, the term “hemostatic agent” refers to any agent that is capable of arresting, stemming, or preventing bleeding. Typically, the agent enhances blot clot formation and more particularly promotes platelet aggregation. The method of the present invention is particularly suitable for accelerating blood clotting. The term is also known as “antihemorrhagic agent”. In particular, LG promotes platelet aggregation. As used herein, the term “platelet aggregation” refers to the attachment of activated platelets one to another, which results in the formation of aggregates or clumps of activated platelets.

As used herein, the term “patient” refers to a mammalian to which the present invention may be applied. Typically said mammal is a human, but may concern other mammals such as primates, dogs, cats, pigs, sheep, cows. In particular, the term “patient” refers to a mammalian patient. In some embodiments, the patient is a human infant. In some embodiments, the patient is a human child. In some embodiments, the patient is a human adult. In some embodiments, the patient is an elderly human.

As used herein, the term “lauryl gallate” or “LG” has its general meaning in the art and refers to the ester of dodecanol and gallic acid. The term is also known as “dodecyl gallate”. The IUPAC name of LG is Dodecyl 3,4,5-trihydroxybenzoate. Commercial sources for LG are well known in the art. The formula of LG is depicted in FIG. 1.

In some embodiments the method of the present invention is particularly suitable in bariatric surgery, cardiac surgery, thoracic surgery, colon and rectal surgery, dermatologic surgery, general surgery, gynecologic surgery, maxillofacial surgery, neurosurgery, obstetric surgery, oncologic surgery, ophthalmologic surgery, oral surgery, orthopedic surgery, otolaryngologic surgery, pediatric surgery, plastic surgery, cosmetic and reconstructive surgery, podiatric surgery, spine surgery, transplant surgery, trauma surgery, vascular surgery, urologic surgery, dental surgery, veterinary surgery, endoscopic surgery, anesthesiology, an interventional radiologic procedure, an emergency medicine procedure, a battlefield procedure, a deep or superficial laceration repair, a cardiologic procedure, an internal medicine procedure, an intensive care procedure, an endocrinologic procedure, a gastroenterologic procedure, a hematologic procedure, a hepatologic procedure, a diagnostic radiologic procedure, an infectious disease procedure, a nephrologic procedure, an oncologic procedure, a proctologic procedure, a pulmonary medicine procedure, a rheumatologic procedure, a pediatric procedure, a physical medicine or rehabilitation medicine procedure, a geriatric procedure, a palliative care procedure, a medical genetic procedure, a foetal procedure, or a combination thereof.

Typically, the hemostatic agent of the invention is applied using conventional techniques. Coating, dipping, spraying, spreading and solvent casting are possible approaches. More particularly, said applying is manual applying, applicator applying, instrument applying, manual spray applying, aerosol spray applying, syringe applying, airless tip applying, gas-assist tip applying, percutaneous applying, surface applying, topical applying, internal applying, enteral applying, parenteral applying, protective applying, catheter applying, endoscopic applying, arthroscopic applying, encapsulation scaffold applying, stent applying, wound dressing applying, vascular patch applying, vascular graft applying, image-guided applying, radiologic applying, brush applying, wrap applying, or drip applying.

As used herein, the term “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the drug are outweighed by the therapeutically beneficial effects. The efficient dosages and dosage regimens for drug depend on severity of bleeding or condition to be treated and may be determined by the persons skilled in the art. A physician having ordinary skill in the art may readily determine and prescribe the effective amount of the pharmaceutical composition required. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of the compound of the present invention is 0.02-100 mg/kg, such as about 0.02-30 mg/kg, such as about 0.05-10 mg/kg or 0.1-3 mg/kg, for example about 0.5-2 mg/kg.

Typically the agent of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some embodiments, in particular, for cutaneous application, the hemostatic agent of the present invention can be deposited on the tissue with means typically selected from the group consisting of a patch, a dressing, or a band-aid having a plurality of capsules (e.g. nanocapsules) having the ability to release the hemostatic agent (when they are contacted by the tissue (e.g. because of a variation of temperature, physical pressure, osmotic pressure . . . ). Then after a while the means can be pull out, and the material or tissue can be approximated with the tissue where the hemostatic agent was adsorbed. Thus, it is generally preferred to deposit the hemostatic agent onto a hemostatic support to yield a hemostatic material to be applied to a bleeding or oozing wound. Hemostatic materials include but are not limited to fabrics, puffs, sponges, sutures, fibers, powder and gels. While it is generally preferred to apply the hemostatic material directly to the wound. To ensure that the hemostatic material remains affixed to the wound, a suitable adhesive can be employed, for example, along the edges of one side of the hemostatic fabric, sponge or puff. Although any adhesive suitable for forming a bond with skin can be used, it is generally preferred to use a pressure sensitive adhesive. Pressure sensitive adhesives are generally defined as adhesives that adhere to a substrate when a light pressure is applied but leave no residue when removed. Pressure sensitive adhesives include, but are not limited to, solvent in solution adhesives, hot melt adhesives, aqueous emulsion adhesives, calenderable adhesive, and radiation curable adhesives. In some embodiments, a composite including two or more layers can be prepared, wherein one of the layers comprises the hemostatic agent of the present invention and another layer is, e.g., an elastomeric layer, gauze, vapor-permeable film, waterproof film, a woven or nonwoven fabric, a mesh, or the like. The layers can then be bonded using any suitable method, e.g., adhesives such as pressure sensitive adhesives, hot melt adhesives, curable adhesives, application of heat or pressure such as in lamination, physical attachment through the use of stitching, studs, other fasteners, or the like.

The expected advantages provided by the method of the present invention include the reduction of blood loss in case of haemorrhage. It is also expected to reduce the adverse effects observed during the administration of blood coagulation factors, especially the risk of thrombosis. Indeed, the method of the present invention is preferably carried out for external local use allowing to reduce the bleeding time and the volume of blood lost contrary to other protocols which require a parenteral administration such as tranexamic acid, fibrinogen and prothrombinic complex concentrates which all expose to a thrombotic risk. A further advantage is that the method of the present invention does not require any medical qualification, especially in case of emergency (civil or military traumatic accidents). The low toxicity and the already known biological activities of LG such as antioxidant, antibacterial and antiviral activities are particularly advantageous for using said compound as an antihemorrhagic agent that can be applied directly on wounds.

The invention will be further illustrated by the following figures and examples. However, these examples and figures should not be interpreted in any way as limiting the scope of the present invention.

### FIGURES

FIG. 1: Chemical structure of Lauryl gallate (LG). LG is the n-alkyl ester of gallic acid, an aromatic organic compound (Kubo et al, 2002).

FIG. 2: Effect of LG on washed platelet aggregation and secretion. (A) Washed platelets from healthy donors were treated or not with LG at the indicated concentrations or vehicle (DMSO) and spontaneous aggregation was assessed by turbidimetry during 15 minutes. Results are expressed as percentage of maximal platelet aggregation and are mean±SEM of 7 independent experiments (*p<0.05; **p<0.01; ****p<0.0001 according to one way ANOVA followed by Sidak's multiple comparisons test). (B) Representative washed platelet aggregation curves are shown. Platelet secretion of (C) a-granules (CD62-P) and (D) dense granules (CD63) was quantified by flow cytometry following LG or vehicle (DMSO) addition during 10 minutes. (E) Activation of the GpIIbIIIA integrin was measured using Pacl antibody by flow cytometry after 10 minutes of LG stimulation. Results are expressed as median fluorescence intensity (MFI) and are mean±SEM of 4 independent experiments (*p<0.05; **p<0.01; ****p<0.0001 according to one way ANOVA followed by Sidak's multiple comparisons test).

FIG. 3: Scanning electron microscopy of platelets following stimulation with LG or TRAP. (A) Representative scanning electron micrographs of washed platelets from healthy donors treated with DMSO (control), TRAP 25 μM, LG 0.05 mM and 0.75 mM during 10 minutes at 37° C. in non-shaking conditions (white scale bar=1 μm). (B) Platelet were stimulated in similar conditions but under shaking conditions to allow platelet aggregation. Images of individual platelets (A) and platelet aggregates (B) are representative of 3 healthy donors.

FIG. 4: Effect of LG on signal transduction events. The effect of LG at 0.75 mM (A) and 0.05 mM (B) on washed human platelet Akt and p38-MAPK activation was analyzed by Western blotting using specific anti-phospho-antibodies (Akt phosphorylation on Ser-473, p38 MAPK phosphorylation on Thr180/Tyr182). The cleavage of Src kinases induced at the two doses of LG was also assessed by Western blotting. Representative Western blots are shown. Quantification was performed by densitometric analysis and results are expressed as fold increase and are mean±SEM of 5 independent experiments (*p<0.05; **p<0.01; ***p<0.001; ****p<0.0001 according to two way ANOVA followed by Sidak's multiple comparisons test).

FIG. 5: Effect of LG on cytosolic calcium concentration assessed using calcifluor 8-AM and flow cytometry. Washed platelets pretreated with CalciFluor 8-AM were stimulated by LG at 0.05 mM (A) or 0.75 mM (B), TRAP 25 μM (C) or CRP 4.5 μg/ml (D) in the presence or not of BAPTA-AM 10 μM, EGTA 1 mM or vehicle (DMSO). Free cytosolic calcium concentration was monitored by flow cytometry during 3 minutes. Results are expressed as cytosolic calcium concentration (nM) and are mean±SEM of 4 independent experiments (*p<0.05; **p<0.01; ***p<0.001; ****p<0.0001 according to one way ANOVA followed by Sidak's multiple comparison test).

FIG. 6: LG potentiates platelet aggregation induced by low doses of collagen or TRAP. Washed platelets were treated with collagen 0.15 μg/ml (A) or TRAP 5 μM (B) and/or LG at 0.2 and 0.3 mM and platelet aggregation was assessed by turbidimetry during 10 minutes. These concentrations of LG were choosen because they induced a weak platelet aggregation (see FIG. 2A). Results, expressed as percentage of maximal aggregation, are mean±SEM of 6 (A) and 5 (B) independent experiments (vehicle versus LG with or without low dose of agonist *p<0.05; **p<0.01; ****p<0.0001; LG alone versus LG with low dose agonists #p<0.05, ##p<0.01 according to one way ANOVA followed by Sidak's multiple comparisons test).

FIG. 7: LG significantly potentiates thrombus formation in whole blood at arterial shear rate. DIOC₆-labeled platelets in whole blood from healthy donors in the presence of LG 0.05 mM or vehicle (DMSO) were perfused through a collagen-coated micro-capillary at a physiological arterial shear rate of 1500 s⁻¹. Surface coverage (A) and thrombi volumes (μm³) (B) were analyzed using ImageJ software. Graphs represent the averages of 3 independent experiments and are mean±SEM (*p<0.05, **p<0.01 according to two way ANOVA followed by Sidak's multiple comparisons test).

FIG. 8: Application of LG strongly decreases the rat tail bleeding time. Immediately after the transection cut, rat tail was dipped for 5 seconds into ethanol (vehicle) or ethanol containing 50 mg/ml of LG. The bleeding time was them indirectly determined using a filter paper to assess blood oozing from the wound without disturbing the forming clot. Tranexamic acid, a known antihemorrhagic solution, was used at a standard concentration of 100 mg/ml in ethanol. Results are expressed in second for the bleeding times. Areas of the first ten drops collected on the filter paper (pixel²) are also provided and results are mean±SEM for 6 independent experiments (vehicle versus LG or Tranexamic acid *p<0.05; **p<0.01; ****p<0.0001; LG versus Tranexamic acid #p<0.05; ##p<0.01 according to one way ANOVA followed by Sidak's multiple comparisons test).

### EXAMPLE

### Material & Methods

### Reagents

Lauryl gallate, ADP, TRAP 14-mer (Thrombin Receptor Activator Peptide 14), prostacyclin and apyrase were obtained from Sigma-Aldrich. HORM collagen type I fibrils from equine tendon (Takeda), BAPTA-AM from Focus bio molecule and calcifluor 8 AM from Santa Cruz Biotechnology. For antibodies, anti-Src family antibody (Tyr416), anti-phospho-Src family antibody, anti-phospho-P38 MAP-kinase and anti-phospho-Akt (Ser 473) were all purchased from Cell signaling. 4G10 Platinium® anti-phospho-tyrosine antibody (Millipore), PAC-1, anti-phospho-CD62 and anti-CD63 were obtained from BD Biosciences and peroxidase-conjugated anti-mouse or anti-rabbit antibodies were from Promega. All other chemicals and reagents used were of the highest commercially available purity.

Lauryl gallate was suspended in dimethyl sulfoxide noted DMSO (LOBA Chemie) at 250 mM as initial concentration. In all experiments, the percentage of DMSO doesn't exceed 0.25%.

### Blood Sampling and Platelets Preparation

Healthy volunteers had abstained from Aspirin and others anti-inflammatory drugs for at least days prior to blood sampling. Written informed consent was obtained from all donors. Blood samples were collected into sodium citrate 3.2% and centrifuged for 10 min at 190 g. The supernatant thus obtained was the platelet-rich plasma (PRP). Further centrifugation at 3500 g for 10 min resulted in platelet-poor plasma (PPP) in the supernatant.

Washed platelets were prepared from centrifuged PRP previously supplemented with rostacyclin (0.5 μM) to avoid platelet activation. The pellet was suspended in buffer A (pH 6.8) containing 140 mM NaCl, 5 mM KCl, 5 mM KH2PO4, 1 mM MgSO4, 10 mM N-2-hydroxyethylpiperazine-N′-2-ethanesulfonic acid, 5 mM glucose and 0.35% bovine serum albumin (w/v). The same buffer containing 1 mM CaCl₂ and apyrase but without BSA (adjusted to pH 7.4) was added to the final suspension to reach 2×10⁸ platelets/ml.

### Platelet Aggregation

Different concentrations of LG were added to 270 μl of PRP or washed platelets from an initial concentration of 250 mM. Platelet aggregation was recorded using a light transmission aggregometer (SD Medical). The percentage of transmittance of PRP or washed platelets was recorded as 0%, and that of the appropriate PPP or buffer was recorded as 100% respectively. Each condition was repeated for many healthy volunteers. Platelets treated with DMSO serves as negative control. Potentiation between low doses of LG and collagen or TRAP to aggregate platelets was also determined.

### Western Blotting

Washed platelets were treated with LG (LC and HC) or DMSO. Aliquots were taken at 0, 2.5, 5 and 10 min, lysed with sample buffer and boiled. Proteins were loaded on to sodium dodecyl sulfate 10% polyacrylamide gel and, after electrophoresis, transferred to a nitrocellulose membrane. Tyrosine phosphorylation was revealed by immunoblotting with the 4G10 Platinium® antiphospho-tyrosine antibody. Phospho-AKT, phospho-P38 MAP kinase and total Src family were revealed by corresponding antibodies. Phospho antibodies were diluted in TBS-Tween 5%-BSA (w/v) and incubated overnight and the others for 2 h. After washing, each blot was incubated with the corresponding horseradish peroxidase-conjugated anti-mouse or anti-rabbit. Immunoblots were developed using Super signal chemiluminescent substrate (Pierce, Rockford, IL) and analyzed with chemidoc Imaging system (Biorad).

### Flow Cytometry Analysis of Platelet-Granules Secretion, αIIbβ3 Activation, and Phosphatidylserine Exposure

Washed platelets were pre-incubated with LG or DMSO for 5 and 10 min at 37° C. Samples were then incubated for 15 min at room temperature in the dark with anti-CD62P, anti-CD63 or monoclonal antibody PAC-1. This last recognize and binds only to the active form of integrin αIIbβ3 complex. Analysis of respective protein was performed using a FACS-Verse cytometer BD Biosciences). Platelets were identified on the basis of their Forward Scatter and Side Scatter properties.

For phosphatidylserine exposure, washed platelets were pre-incubated with LG 0.05 mM or DMSO for 5 min at 37° C. Dilute the activated or non-activated sample with the 1× binding buffer provided in the kit to a final volume of 100 μl and add 5 μl of Annexin V AF647 (Clinisciences) for 15 min at 37° C. in the dark. The binding reaction is stopped with 500 ill of 1× binding buffer (Ramstrom 2010). Analysis was performed using LSR Fortessa cytometer (BD Biosciences).

### Thrombus Formation on Immobilized Collagen Under Flow Conditions

This method was performed as previously described (Gratacap et al, 2009). Briefly, biochips with micro-capillaries (Vena8Fluoro+®, Cellix) were coated with collagen type I. Epi-fluorescence microscope (Axio Observer; Carl Zeiss) allowed direct visualization of platelet adhesion and aggregation which was recorded with ORCA-R2 camera (Hamamatsu). Whole blood was collected into 13.6 UI/m1 lithium heparinate and platelets were labeled with DIOC6(Life Technologies, 2 μM, 10 min at 37° C.) LG (0.05 mM) was then added to blood just prior to injection through a micro-capillary at shear rate of 1500 s⁻¹. Thrombus formation was visualized with a 40× long working distance objective in real time. The range was fixed to 80 μm and the interval to 1.5 μm. The acquisition rate was chosen at one frame every 30 sec divided into two positions. Quantification of surface coverage was performed off-line using ImageJ software.

### Calcium Mobilization

Platelet calcium flux was adapted from the work described by (Monteiro et al. 1999). Washed platelets re-suspended at 10⁸/ml in platelet wash buffer were incubated with the calcium indicator dyes Calcifluor 8-AM (R&D system) at 5 μM and Pluronic F-127 0.02% v/v (Sigma Aldrich) for 30 min at 37° C. The platelets were washed twice with platelet wash buffer, re-suspended in Tyrode's buffer containing EGTA (1 mM) or BAPTA-AM (10 μM). Changes in cytosolic calcium concentration were associated with increased or decreased fluorescence emission of Calcifluor 8-AM (emission wavelengths of 500-570 nm). These changes were analyzed using flow cytometry BD LSRII Fortessa. Results were expressed in fold increased Median Fluorescence. The determination of internal calcium concentration in platelet was described by Grynkiewicz et al. (1985).

### Scanning Electron Microscopy.

Platelets are treated with TRAP 25 μM, LG (LC and HC) during 10 minutes. They were fixed in 2.5% glutaraldehyde in 0.1 M Sorensen phosphate buffer (pH 7.4) for at least 1 h at 37° C. After sedimentation, the pellets were re-suspended in water and made to adhere on poly-lysine coated glass coverslips. Platelets were then dehydrated in a graded ethanol series and dried by critical point drying with a Leica EM CPD 300. The samples were coated with 6 nm Platinium on a Leica EM Med 020 before being examined on a FEI Quanta 250 FEG scanning electron microscope, at an accelerating voltage of 5 kV (Valet 2021).

### Measurement of LDH Leakage

Washed platelets from healthy donors were treated with different doses of LG or control (DMSO) for 10 minutes at 37° C. Following, platelets were pelleted by centrifugation at 1800×g for 3 min. Supernatants were used to detect Lactate dehydrogenase (LDH) release by colorimetric method (Thermo Fisher Scientific), according to the manufacturer's protocol. The assay was performed in endpoint time of increased of formazan absorbance at 680 nm for 30 min. Maximal LDH leakage was obtained with lysis buffer. Results, expressed as percentage of LDH leakage are mean±SEM (n=6) (** p<0.01, *** p<0.001 according to Kruskal-Wallis test).

### Rat Tail Bleeding Time

Wistar rats of either gender weighing between 200 and 250 g were purchased from the Central Pharmacy of Tunisia. Animal handling conforms to the European Convention (CE. no. 123). Animals were fed a standard chow diet and had free access to water. They were housed five rats in a cage (0.12 m²). Bleeding time was measured by transection of the tail, 1 cm from the tip, of phenobarbital anesthetized rats (25 mg/kg. i.p). Immediately after transection, the tail was dipped for 5 seconds into LG solution (5, 50, or 100 mg/ml of LG dissolved in ethanol) or vehicle (ethanol) and then placed carefully on Whatman paper. Blood was allowed to drop on filter paper which was moved gently every 30 seconds until no more blood was dropped. If bleeding did not resume within 30 seconds of cessation, it was considered stopped (Bastaki et al, 2017). The bleeding time was also determined for a lot of rats not treated with any solution (control) and for another treated with ethanol to test the effect of the solvent. Tranexamic acid (100 mg/ml) was used as a standard anti-hemorrhagic drug under the same conditions. Each condition was repeated for six rats.

### Statistical Analysis

Results were presented as the mean±SEM of at least three independent experiments. Statistical significance among groups was analyzed by Two-way ANOVA test, Mann Whitney test or Kruskal-Wallis test using Graph pad prism program [p<0.05 (*); p<0.01 (**); p<0.001 (***); p<0.0001 (****)].

### RESULTS

### Lauryl Gallate Induces Platelet Aggregation, Granules Secretion and Phosphatidylserine Exposure

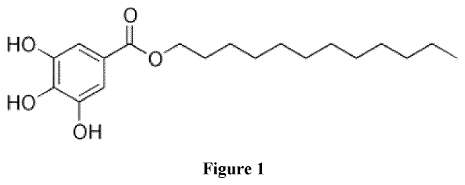

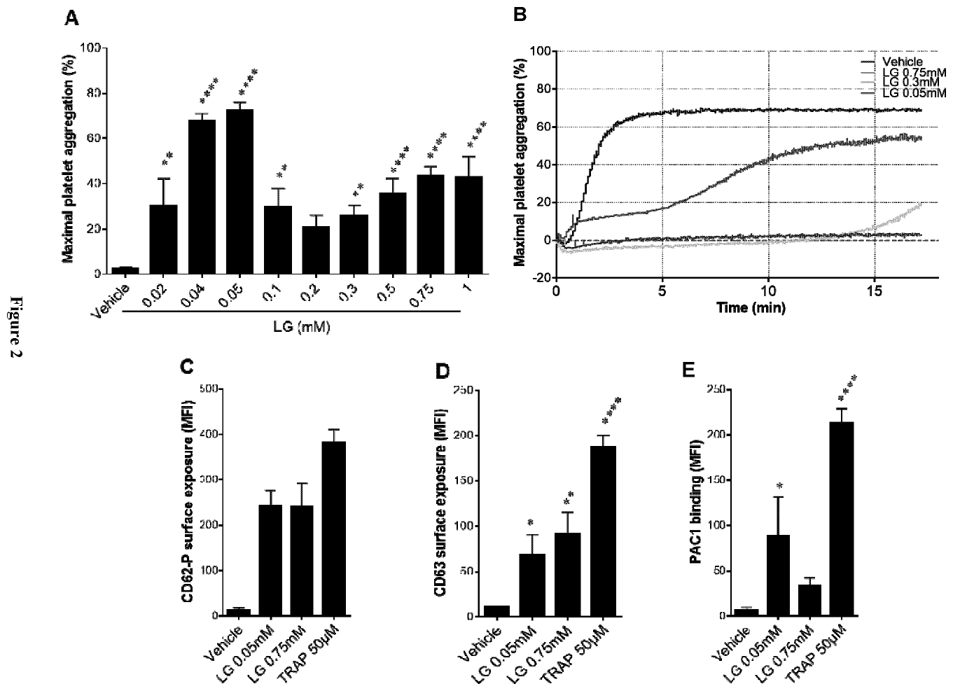

LG, the ester of dodecanol and gallic acid (FIG. 1), was first tested alone at increasing concentrations on washed human platelet aggregation response. Interestingly, as shown in FIG. 2A, LG induced a non-monotonic dose-response with the first peak of platelet aggregation response at low concentration (0.05 mM) followed by a decreased platelet aggregation response at higher concentrations (until 0.2 mM) and again an increase in maximal platelet aggregation response at high doses of LG reaching a plateau at 0.75 to 1 mM. Platelet aggregation induced by 0.05 mM LG was fast, reached nearly 80% of maximal aggregation and remained stable (FIG. 2B). The aggregation trace was comparable to that classically obtained following stimulation by a physiological platelet agonist. In contrast, a slow platelet aggregation response, hardly reaching 50% of maximal aggregation, was observed when high concentrations of LG were used (FIG. 2B).

The surface expression of the platelet α-granule secretion marker CD62-P (P-selectin) and the dense granules secretion marker CD63 was assessed by flow cytometry. The expression of CD62-P on the plasma membrane of washed platelets was markedly increased following the addition of LG at 0.05 mM or at 0.75 mM. For the sake of comparison, LG-induced a-granule secretion intensity was slightly lower than that induced by 50 μM of thrombin receptor agonist peptide (TRAP). The plasma membrane expression level of CD63 was also significantly increased following the addition of LG indicating that platelets were activated and secreted both their alpha and dense granules (FIGS. 2C and 2D). In addition, washed platelets stimulated by LG showed a significant increase in the expression of the active form of the fibrinogen receptor αIIbβ3, particularly at the low dose of 0.05 mM (FIG. 2E). The activation of αIIbβ3 by LG at 0.05 mM was however 50% lower than that induced by 50 μM TRAP. Moreover, we investigated whether LG would induce procoagulant platelet formation by measuring annexin V binding to phosphatidylserine (PS) exposed on the outer leaflet of the platelet plasma membrane. Interestingly, LG at 0.05 mM induced a significant exposure of PS on the platelet surface by annexin V-FITC binding (data not shown).

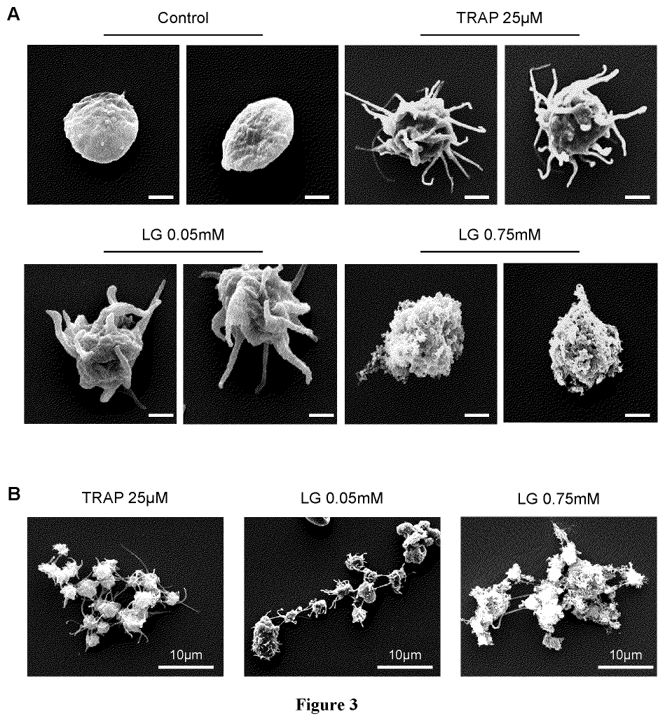

Taken together, these data demonstrate that LG induced spontaneous platelet activation, secretion, and aggregation. However, the profile of aggregation response was different when low (0.05 mM) or high (0.75 mM) doses of LG were used, suggesting a different mechanism of platelet activation. Knowing that LG could disturb platelet membrane organization we analyzed the morphology of washed platelets treated with LG at 0.05 mM and 0.75 mM by scanning electron microscopy. As expected, resting platelets from healthy donors were discoid while TRAP-stimulated platelets, in non-shaking condition, changed their shape and emitted filopodia (FIG. 3A). Interestingly, LG at 0.05 mM also induced platelet shape change and filopodia formation, although the platelet body was less contracted and filopodia were thicker than TRAP-stimulated platelets. At high concentration of LG (0.75 mM), platelets also changed their shape but filopodia were no longer visible and the membrane structure was strongly modified with a grainy appearance and apparent pores formation (FIG. 3A). Under shaking conditions, platelet aggregates were formed in all cases with again different morphology of platelets (FIG. 3B). Platelet aggregates induced by LG at 0.75 mM are very different from those induced by TRAP or LG at 0.05 mM.

Potential cell membrane damage was further investigated by measuring LDH leakage following treatment of washed platelet with increasing doses of LG. At low doses of LG 0.1 mM), no significant LDH release from platelets could be detected which is consistent with the intact membrane structure observed by electron microscopy (data not shown). However, at higher doses of LG (>0.5 mM), a significant percentage of LDH (between 10 and 20% of total LDH) was detected in the supernatant confirming that LG induced platelet membrane damage and porosity as shown in the scanning electron micrographs (data not shown).

### LG Triggers Signal Transduction Pathways in Human Platelets

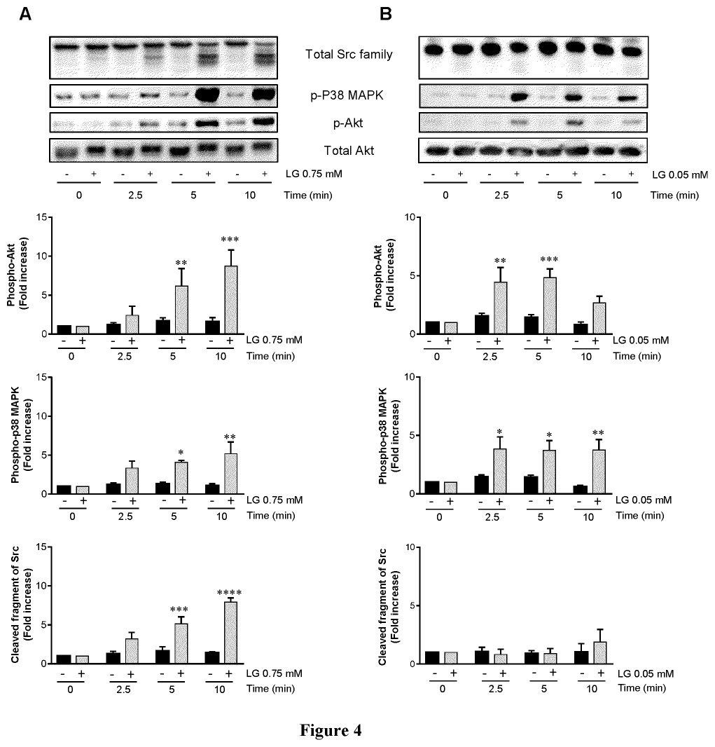

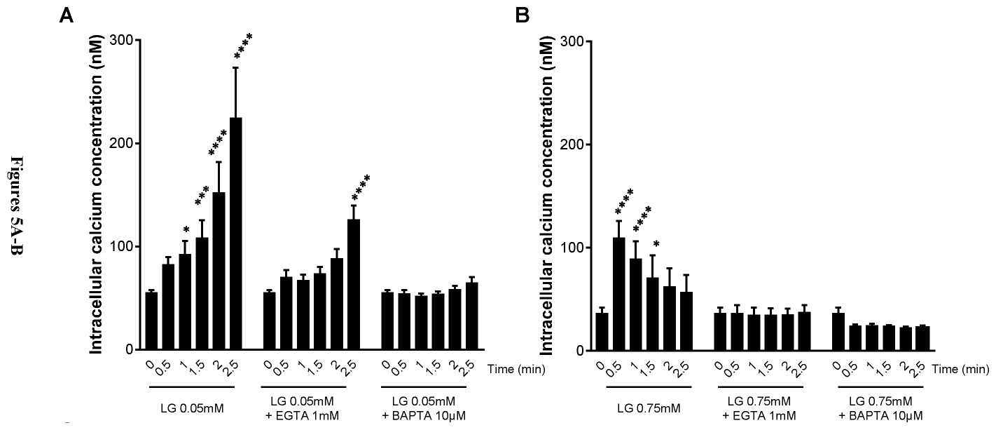

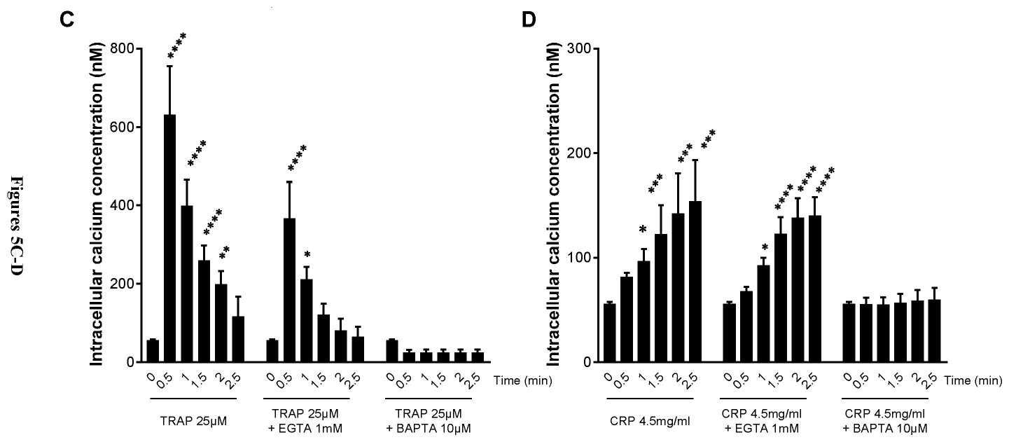

Having shown that platelets form aggregates following LG addition, we next investigated if intracellular signal transduction pathways would be activated. We first investigated the phosphorylation of Akt at Ser473 and of p38 MAPK at Thr180/Tyr182 by Western blotting, as well as the cleavage of Src kinases. Src-kinases are known to be cleaved by calpains, a family of protease activated by high cytosolic calcium concentration, at their N-terminal domain generating a truncated Src fragment of ˜52 kDa. Consistent with the rapid induction of platelet aggregation, 0.05 mM of LG induced a rapid activation of Akt and p38 MAPK phosphorylation (FIG. 4B). The high dose of LG (0.75 mM) also induced Akt and p38 MAPK activation but at later times corroborating the late platelet aggregation observed in this condition (FIG. 4A). In parallel, while the low dose of LG had no impact on Src-kinase cleavage, at 0.75 mM LG induced a cleavage of Src-kinase which was substantial at 10 min (FIGS. 4A and B). These data indicate that LG is able to trigger intracellular platelet signaling pathways with differences in velocity and Src-kinase cleavage intensity at low and high concentrations. To further characterize LG-induced intracellular signaling pathways we analyzed calcium signaling using CalciFluor (Fluo-8) to label free cytosolic calcium and flow cytometry detection. The effect of LG on washed human platelets was compared to that induced by 25 μM TRAP or 4.5 μg/ml collagen-related peptide (CRP). LG at 0.05 mM induced a robust increase of cytoplasmic free calcium concentration (reaching 230±25 nM after 2.5 min) comparable in kinetic and intensity to that induced by CRP (FIG. 5A). This increase in cytosolic free calcium concentration was blocked by the intracellular calcium chelator BAPTA-AM and partly inhibited by addition of EGTA indicating that the low dose of LG induced both a calcium mobilization and a calcium influx. Addition of LG at 0.75 mM induced a rapid increase in free cytosolic calcium reaching 105±15 nM at 0.5 min that decreased thereafter over time (FIG. 5B). This calcium increase was totally abrogated by addition of both BAPTA-AM and EGTA indicating that it was largely due to calcium influx. Of note, the flow cytometry analysis indicated the appearance of a platelet subpopulation which had lost the fluorescent probe suggesting that their membranes were leaky following addition of the high dose of LG, as also pointed by electron microscopy and LDH measurements.

As expected, TRAP induced a rapid and transient calcium responses that was due to both calcium mobilization and influx (FIG. 5C) while CRP initiated a slower but sustained calcium response largely due to calcium mobilization (FIG. 5D).

LG strongly potentiates platelet aggregation and thrombus formation in whole blood under flow. We then checked whether LG would potentiate platelet aggregation induced by low doses of collagen (0.15 μg/ml) or TRAP (5 μM). Two doses of LG (0.2 and 0.3 mM) that were poorly efficient to trigger washed platelet aggregation were used. While 0.15 μg/ml of collagen and 5 μ M of TRAP alone were unable to trigger detectable platelet aggregation, the association with

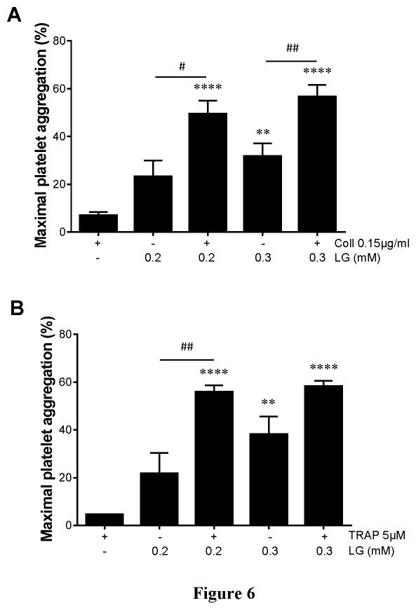

LG induced a significant potentiation of the response reaching 50 to 60% of the maximal platelet aggregation (FIGS. 6A and B).

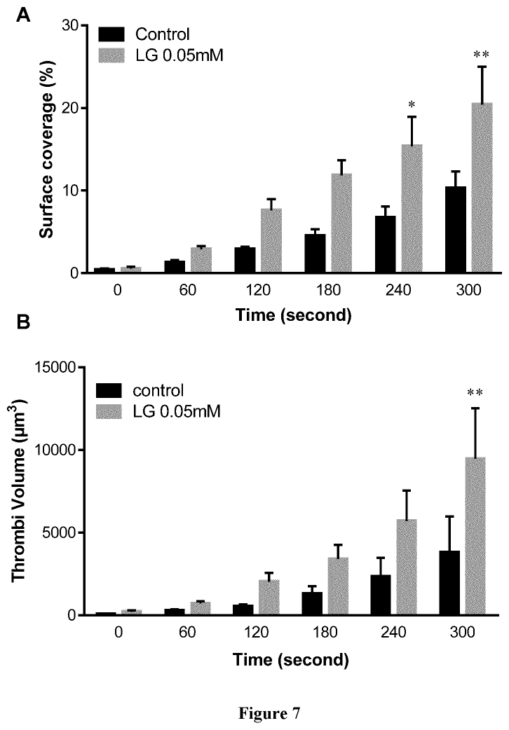

To further investigate the effect of LG at low doses (0.05 mM) on hemostasis, we performed a microfluidic-based thrombus formation assay using whole blood perfused at physiological arterial shear stress on collagen-coated microcapillaries. Platelets in human whole blood were labeled with DIOC6, and accumulation of fluorescent platelets on collagen-coated surface was recorded by a video microscopy system allowing to quantify the surface covered by platelets and the thrombus volume. Addition of 0.05 mM of LG to the blood significantly increased thrombus volumes compared to control (FIG. 7). Of note, in several experiments the effect of LG on thrombus formation was so strong that the capillaries were occluded before the end of the recording (300 s).

### LG Application Strongly Reduces the Tail Bleeding Time in Rats

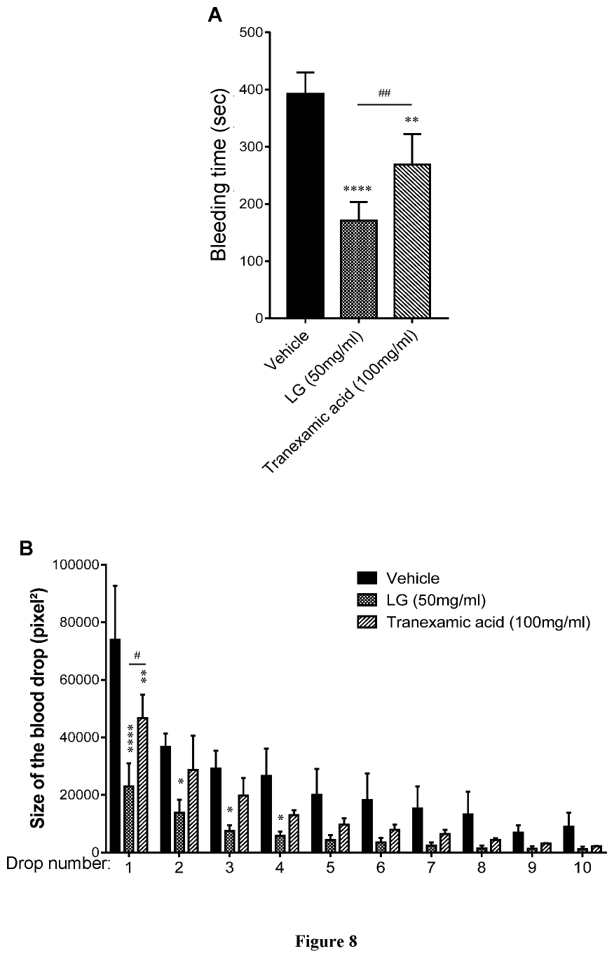

To assess the hemostatic action of LG in vivo we performed a tail bleeding assay in rats. After section, the tail was dipped in LG solution for 5 seconds and the bleeding time was evaluated by gently dabbing with a Whatman paper until bleeding stopped. Tranexamic acid, an antifibrinolytic compound well-known to reduce bleeding, was used for sake of comparison. Interestingly, LG application dramatically reduced the bleeding time and was even significantly more efficient than tranexamic acid on this murine bleeding time model (FIG. 8).

### DISCUSSION

LG is widely used in food industry as antioxidant, specially to prevent oxidative rancidity of foods containing fats and oils (Kubo et al, 2002). Following the discovery of its antibacterial and antiviral actions, numerous other functions were reported including anti-proliferative and anti-tumor activities (Ortega et al, 2003) (Teng et al, 2014). Here we demonstrate that LG can induce human platelet shape change, filopodia formation, granule secretion, PS exposure and platelet aggregation. At low dose of LG (0.05 mM), the maximal platelet aggregation was reached quickly and the aggregation remained stable and comparable to that induced by physiological agonists. LG-induced platelet activation was associated with the secretion of both α and dense granules and with the activation of the αIIbβ3 integrin, an essential step allowing platelet aggregation through fibrinogen binding. These platelet responses were linked to a significant activation of intracellular signal transduction pathways including, phosphorylation of Akt, a well-known effector of class 1 phosphoinositide 3-kinase (Ribes et al, 2020) and p38MAPK activation (Mazharian et al, 2005). This rapid activation of signaling proteins was accompanied by a significant cytosolic calcium response. Indeed, LG at low dose induced a rapid rise in cytosolic calcium which was due to both calcium mobilization and calcium influx. This calcium response was comparable in terms of kinetics and intensity to that initiated by the GPVI agonist CRP which is however mainly due to calcium mobilization. Intracellular free calcium elevation is central to platelet activation as it controls many molecular mechanisms including phospholipase A2 activation, scramblase activity or cytoskeleton organization and in turn key responses such as granule secretion, platelet aggregation and procoagulant activity (Harper and Sage, 2017).

However, it is important to note that at high concentration (0.75 mM) LG has a different effect on platelets. It induced a dramatic membrane modification as shown by scanning electron microscopy with a grainy appearance and apparent pores formation as also suggested by LDH measurement. These effects are consistent with the hydrophobic nature of LG which has been shown to insert into biological membranes and to modify their organization (Jurak and Minones 2016). Platelet aggregation induced by the high concentration of LG was slow with a maximal aggregation hardly reaching 50%. Contrary to the low dose of LG, in most cases platelets did not extend filopodia and the rise in cytosolic calcium was mainly due to calcium influx. This was accompanied by an important cleavage of Src-kinases with the generation of a truncated Src fragment known to be generated by calcium-dependent calpain family members. At the low dose, LG was able to trigger platelet activation without visible membrane modification by electron microscopy or LDH release. Platelet activation induced by the low dose of LG resemble that induced by physiological agonist suggesting that it could activate platelet receptors either by interaction or more likely by modifying the fluidity of the plasma membrane leading to receptor clustering and activation independently of ligand. Change in membrane fluidity will modify the capacity of lipids and proteins to diffuse laterally in the plane of the membrane and may induce lipid rafts coalescence and clustering of membrane receptors (Simons et al 2011). The non-monotonic dose-response of LG on platelet aggregation response and our electron microscopy data are consistent with such a membrane effect of LG on platelets.

Moreover, close to LG, propyl gallate, an ester formed by the condensation of gallic acid and propanol also used in food preservation has been shown to induce platelet aggregation (Hongyan and Richard, 2004) possibly by modifying platelet membrane (Qi and Hu, 1993).

Interestingly, LG was not only active on washed human platelets, since addition of a low dose of LG (0.05 mM) to human whole blood strongly increased the thrombotic response of platelet on collagen matrix under normal arterial shear rate. The surface covered by platelets and the volume of the platelet thrombi formed on the collagen matrix in the microfluidic system were significantly increased in the presence of LG. This result is consistent with the fact that LG potentiated washed platelet responses induced by subthreshold doses of collagen or TRAP suggesting synergy of action. These result stimulated us to investigate the impact of a flash LG application on the tail bleeding time in rats. In agreement with the results obtained in vitro in the microfluidic system, LG application after tail section significantly reduced the bleeding time. LG was even significantly more efficient that the antifibrinolytic agent tranexamic acid on this murine bleeding time model. These data suggest that LG has hemostatic and antihemorrhagic potential when applied to a wound. Although the concentration of LG that may be found in the blood after ingestion is not documented, one can also suggest a potential increased risk of thrombosis after important intake of this food additive.

In conclusion, this study shows for the first time that the food additive LG (E312) has potent effects on washed human platelets and strongly potentiate platelet thrombus formation in whole blood under arterial shear rate. LG activates platelet intracellular signaling, particularly calcium signaling, possibly by modifying membrane lateral organization. Its hemostatic and antihemorrhagic properties may suggest a potential therapeutic use of this molecule.

### REFERENCES

Throughout this application, various references describe the state of the art to which this invention pertains. The disclosures of these references are hereby incorporated by reference into the present disclosure.
Antonio Serrano, Carmen Palacios, Garbin\tilde{ }e Roy, ConstantinoCespo\acute{ }n, MariBa L. Villar, Mercedes Nocito, and Pedro Gonza\acute{ }lez-Porque\acute{ }. Derivatives of Gallic Acid Induce Apoptosis in Tumoral Cell Lines and Inhibit Lymphocyte Proliferation. ARCHIVES OF BIOCHEMISTRY AND BIOPHYSICS Vol. 350, No. 1, February 1, pp. 49-54, 1998 Blood Coagul Fibrinolysis. 2004; 15 (3):199-206.
Gratacap M P, Martin V, Valéra M C, Allart S, Garcia C, Sié P, Recher C, and Bernard Payrastre. The new tyrosine-kinase inhibitor and anticancer drug dasatinib reversibly affects platelet activation in vitro and in vivo. Blood (2009):114(9), 1884-1892
Grynkiewicz G, Poenie M, and Tsien R Y. A New Generation of Ca2+ Indicators with Greatly Improved Fluorescence Properties. The Journal of biological chemistry (1985):260(6), 3440-3450
Grynkiewicz G, Poenie M, Tsien R Y. A new generation of Ca2+ indicators with greatly improved fluorescence properties. J Biol Chem. 1985 Mar. 25; 260 (6):3440-50.
Hurtado Cl, Bustos M J, Sabina P, Nogal M L, Granja A G, González M E, Gónzalez-Porqué P, Revilla Y, Carrascosa A L. Antiviral activity of lauryl gallate against animal viruses. AntivirTher. 2008; 13 (7): 909-17.
Jurak M and Minones Jr J. Interaction of lauryl gallate with phospholipid components of biological membranes. Biochimica Biophysic Acta 1858 (2016) 1821-1832
Kontogiorgis C, Nicolotti O, Mangiatordi G F, Tognolini M, Karalaki F, Giorgio C, Patsilinakos A, Carotti A, Hadjipavlou-Litina D, Barocelli E. (2015). Studies on the antiplatelet and antithrombotic profile of anti-inflammatory coumarin derivatives. Journal of enzyme inhibition and medicinal chemistry, 30, 925-933.
Kubo Isao, Fujita K, Nihei K, Masuoka N. Non-antibiotic Antibacterial activity of Dodecyl gallate. Bioorg Med Chem (2003) 11: 573-580
Kubo I, Masuoka N, Xiao P, Haraguchi H. Antioxidant activity of dodecyl gallate. J Agric. Food. Chem. 50 (2002) 3533-3539
Levy-Toledano S. Platelet signal transduction pathways: could we organize them into a ‘hierarchy’ (1999). Haemostasis 29(1):4-15.
Leytin V, Mody M, Semple J W, Garvey B, Freedman J. Quantification of platelet activation status by analyzing P-selectin expression. Biochem Biophys Res Commun. 2000 5; 273(2):565-70.
Li Z, Zhang G, Le Breton G C, Gao X, Malik A B, Du X. Two waves of platelet secretion induced by thromboxane A2 receptor and a critical role for phosphoinositide 3-kinases. J Biol Chem. (2003) 15; 278(33):30725-31.
Mahata S, Maru S, Shukla S, Pandey A, Mugesh G, Das B C and Bharti A C. (2012). Anticancer property of Bryophyllumpinnata (Lam.) Oken.leaf on human cervical cancer cells. BMC Complementary and Alternative Medicine 12:15
Maria doCeuMonteiro, Filipe Sansonetty, Maria Jose Goncalves and Jose-Enrique O'Connor Flow Cytometric Kinetic Assay of Calcium Mobilization in Whole Blood Platelets Using Fluo-3 and CD41 Cytometry 35:302-310 (1999).
Murakami T1, Komiyama Y, Masuda M, Kido H, Nomura S, Fukuhara S, Karakawa M, Iwasaka T, Takahashi H. Flow cytometric analysis of platelet activation markers CD62P and CD63 in patients with coronary artery disease. Eur J Clin Invest. (1996) 26(11):996-1003.Ortega E, Sadaba M C, Ortiz A1, Cespon C, Rocamora A, Escolano J M, Roy G, Villar L M and Gonzalez-Porque P. Tumoricidal activity of lauryl gallate towards chemically induced skin tumours in mice. British Journal of Cancer (2003) 88, 940-943
Ruggeri Z M, Mendolicchio G L. Adhesion Mechanisms in Platelet Function. Circ Res. (2007) 100: 1673-1685
Shih-Sheng Chang,1, 2 Viola S. Y. Lee,1 Yu-Lun Tseng,1, 3 Kuan-Cheng Chang,2 Kuen-Bao Chen,4 Yuh-Lien Chen,5 and Chi-Yuan Li1, Gallic Acid Attenuates Platelet Activation and Platelet-Leukocyte Aggregation: Involving Pathways of Akt and GSK3β; Evidence-Based Complementary and Alternative Medicine Volume 2012, Article ID 683872, 8 pages doi: 10.1155/2012/683872
Stegner D & Nieswandt B, Platelet receptor signaling in thrombus formation. (2011) J Mol Med (2011) 89:109-121
Stejskal D, Václavik J, Lacnák B, Prosková J. Aspirin resistance measured by cationic propyl gallate platelet aggregometry and recurrent cardiovascular events during 4 years of follow-up. Eur J Intern Med. 2006;17(5):349-54.
Su W, Chen Y, Wang C, Ding X, Rwibasira G, Kong Y. Human cathelicidin LL-37 inhibits platelet aggregation and thrombosis via Src/PI3K/Akt signaling. BiochemBiophys Res Commun. (2016) 473(1):283-289.

T. J. Rink and S. O. Sage, “Calcium signaling in human platelets,” Annual Review of Physiology, vol. 52, pp. 431-449, 1990
Teng C L, Han S M, Wu W C, Hsueh C M, Tsai J R, Hwang W L and Hsu S L: Mechanistic aspects of lauryl gallate-induced differentiation and apoptosis in human acute myeloid leukemia cells. Food Chem Toxicol 71: 197-206, 2014
van der Heijden, C A., Janssen, P J., Strik, J J 1986. Toxicology of gallates: a review and evaluation. Food Chem. Toxicol
WenSu, Yahui Chen, Caihui Wang, Xue Ding, GamarielRwibasira, Yi Kong. Human cathelicidin LL-37 inhibits platelet aggregation and thrombosis via Src/PI3K/Aktsignaling.
Biochemical and Biophysical Research Communications Volume 473, Issue 1, 22 Apr. 2016, Pages 283-289
Xiao H, Kovics R, Jackson V, Remick D G. Effects of platelet inhibitors on propyl gallate-induced platelet aggregation, protein tyrosine phosphorylation, and platelet factor 3 activation. Zhenyu Li 1, M Keegan Delaney, Kelly A O'Brien, Xiaoping Du Signaling during platelet adhesion and activation. ArteriosclerThrombVascBiol 2010 December; 30(12):2341-9.

1. A method of arresting the flow of blood from a bleeding wound in a patient in need thereof comprising the step of applying a therapeutically effective amount of lauryl gallate (LG).

2. The method of claim 1 for use in bariatric surgery, cardiac surgery, thoracic surgery, colon and rectal surgery, dermatologic surgery, general surgery, gynecologic surgery, maxillofacial surgery, neurosurgery, obstetric surgery, oncologic surgery, ophthalmologic surgery, oral surgery, orthopedic surgery, otolaryngologic surgery, pediatric surgery, plastic surgery, cosmetic and reconstructive surgery, podiatric surgery, spine surgery, transplant surgery, trauma surgery, vascular surgery, urologic surgery, dental surgery, veterinary surgery, endoscopic surgery, anesthesiology, an interventional radiologic procedure, an emergency medicine procedure, a battlefield procedure, a deep or superficial laceration repair, a cardiologic procedure, an internal medicine procedure, an intensive care procedure, an endocrinologic procedure, a gastroenterologic procedure, a hematologic procedure, a hepatologic procedure, a diagnostic radiologic procedure, an infectious disease procedure, a nephrologic procedure, an oncologic procedure, a proctologic procedure, a pulmonary medicine procedure, a rheumatologic procedure, a pediatric procedure, a physical medicine or rehabilitation medicine procedure, a geriatric procedure, a palliative care procedure, a medical genetic procedure, a foetal procedure, or a combination thereof.

3. The method of claim 1 wherein LG is applied by manual applying, applicator applying, instrument applying, manual spray applying, aerosol spray applying, syringe applying, airless tip applying, gas-assist tip applying, percutaneous applying, surface applying, topical applying, internal applying, enteral applying, parenteral applying, protective applying, catheter applying, endoscopic applying, arthroscopic applying, encapsulation scaffold applying, stent applying, wound dressing applying, vascular patch applying, vascular graft applying, image-guided applying, radiologic applying, brush applying, wrap applying, or drip applying.

4. The method of claim 1 wherein LG is deposited on the tissue with means typically selected from the group consisting of a patch, a dressing, or a band-aid having a plurality of capsules (e.g. nanocapsules) having the ability to release the hemostatic agent.

5. A hemostatic material comprising an amount of LG.

6. A method of arresting the flow of blood from a bleeding wound in a patient in need thereof comprising the step of applying a pharmaceutical composition comprising an amount of LG and a pharmaceutically acceptable excipient to the wound.

7. The method according to claim 6 wherein the pharmaceutically acceptable excipient is ethanol.

8. (canceled)
